SEQUENCE LISTING (15 sequences)

SEQ ID NO: 1 - DNA, 19 bp, Artificial Sequence
  forward PCR primer for TGF-B 1
gctgctgacccccactgat

SEQ ID NO: 2 - DNA, 18 bp, Artificial Sequence
  reverse PCR primer for TGF-B 1
gccactgccggacaactc

SEQ ID NO: 3 - DNA, 25 bp, Artificial Sequence
  PCR probe for TGF-B 1
cgcctgagtggctgtcttttgacgt

SEQ ID NO: 4 - DNA, 23 bp, Artificial Sequence
  forward PCR primer for SMA
tcctgaccctgaagtatccgata

SEQ ID NO: 5 - DNA, 22 bp, Artificial Sequence
  reverse PCR primer for SMA
ggtgccagatcttttccatgtc

SEQ ID NO: 6 - DNA, 25 bp, Artificial Sequence
  PCR probe for SMA
aacacggcatcatcaccaactggga

SEQ ID NO: 7 - DNA, 21 bp, Artificial Sequence
  forward PCR primer for Collagen I
ttcacctacagcacgcttgtg

SEQ ID NO: 8 - DNA, 22 bp, Artificial Sequence
  reverse PCR primer for Collagen I
gatgactgtcttgccccaagtt

SEQ ID NO: 9 - DNA, 21 bp, Artificial Sequence
  PCR probe for Collagen I
atggctgcacgagtcacaccg

SEQ ID NO: 10 - DNA, 19 bp, Artificial Sequence
  forward PCR Primer for Fibronectin
ccaaagccaccggagtctt

SEQ ID NO: 11 - DNA, 20 bp, Artificial Sequence
  reverse PCR Primer for Fibronectin
gcttgaagccaatccttgga

SEQ ID NO: 12 - DNA, 29 bp, Artificial Sequence
  PCR probe for Fibronectin
ctctgcgctccattccaccttataacacc

SEQ ID NO: 13 - DNA, 19 bp, Artificial Sequence
  Forward primer for TIMP1
gaaccgcagcgaggagttt

SEQ ID NO: 14 - DNA, 21 bp, Artificial Sequence
  Reverse primer for TIMP1
ggcagtgatgtgcaaatttcc

SEQ ID NO: 15 - DNA, 22 bp, Artificial Sequence
  Probe for TIMP1
tcatcgcgggccgtttaaggaa

(19) United States
(12) Patent Application Publication
(10) Pub. No.: US 2007/0015796 A1
(43) Pub. Date: Jan. 18, 2007

(54) Compositions and methods for treatment of fibrosis

(75) Inventors: Stacey Ann Jones, Durham, NC (US); Yaping Liu, Durham, NC (US); John Tomlin Moore, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: SMITHKLINE BEECHAM CORPORATION, Philadelphia, PA (US)

(21) Appl. No.: 10/572,974

(22) PCT Filed: Sep. 10, 2004
(86) PCT No.: PCT/US04/29748

§ 371 Date: Mar. 22, 2006

Related U.S. Application Data
(60) Provisional application No. 60/506,394, filed on Sep. 26, 2003.

(51) Int. Cl. A61K 31/4439; A61K 31/42

(52) U.S. Cl. 514/340; 514/378

(57) ABSTRACT

Methods for the treatment of fibrosis, including liver fibrosis, via administration of FXR agonists are provided.

### FIELD OF THE INVENTION

The present invention relates to the use of Farnesoid X Receptor (FXR) agonists in the treatment of fibrosis, including liver fibrosis.

### BACKGROUND OF THE INVENTION

The human body responds to trauma and injury by scarring. Fibrosis, a disorder characterized by excessive scarring, is thought to be the result of the normal wound healing response gone awry. One hallmark of fibrosis is the excessive production and deposition of collagen and other extracellular matrix components. Causes of fibrosis are diverse, and include trauma, surgery, infection, and exposure to toxins (including environmental pollutants, alcohol and other toxins). Fibrosis is also associated with various disease states such as diabetes, obesity and non-alcoholic steatohepatitis. Fibrotic disorders can be characterized as acute or chronic, but share the common characteristic of excessive collagen accumulation and an associated loss of function as normal tissue is replaced or displaced by fibrotic tissue. Organs that are commonly affected by fibrosis include liver, kidney and lung. In one sense, fibrosis is not a distinct disease, but is a histological response to other disease processes such as inflammation.

Farnesoid X receptor (FXR) is a member of the nuclear receptor superfamily of ligand activated transcription factors (Lu et al. J. Biol. Chem. 2001 17:17). FXR (also known as RIP14 and NR1H4) is reported to bind and be activated by a variety of naturally occurring bile acids, including the primary bile acid chenodeoxycholic acid and its taurine and glycine conjugates (Makishima et al. Science 1999 284:1362-5; Parks et al. Science 1999 284:1365-8; and Wang et al. Mol. Cell. 1999 3:543-53). A number of recent studies have implicated FXR in the regulation of genes encoding proteins involved in the biosynthesis and transport of bile acids (Sinal et al. Cell 2000 102:731-44; Lu et al. Mol. Cell 2000 6:507-15; Goodwin et al. Mol. Cell. 2000 6:517-26; Grober et al. J. Biol. Chem. 1999 274:29749-54).

FXR ligands have been suggested for use in modulating cholesterol metabolism as well as other disorders. See, e.g., published US patent application US20030003520, US Pat. No. 6,465,258; WO03/030612; WO03/016288; WO03/016280; WO 02/1020463; WO 03/015777; WO 03/015771; and PCT/US03/10519.

In view of the morbidity and mortality caused by fibrotic disorders, the identification of novel methods of treating fibrosis would be beneficial.

### SUMMARY OF THE INVENTION

A first aspect of the present invention is a method for treating organ fibrosis in a mammal, by administering to the subject a therapeutically effective amount of an FXR agonist.

A further aspect of the present invention is a method of reducing or preventing the development of organ fibrosis in a mammal, by administering to a subject in need of such treatment a therapeutically effective amount of an FXR agonist.

### BRIEF DESCRIPTION OF THE FIGURE

FIG. 1A graphs the level of alpha SMA mRNA in livers of groups of Bile Duct Ligated (BDL) rats, where the first column is sham-operated rats, second column is BDL rats treated with vehicle, third column is BDL rats treated with the FXR agonist GW4064, and fourth column is BDL rats treated with taurine-conjugated ursodeoxycholic acid (TUDCA). The asterisk (*) indicates that the level of alpha SMA mRNA in livers of vehicle-, GW4064-, or TUDCA-treated BDL rats is significantly different (p<0.05) than the level in sham operated rats.

FIG. 1B graphs the level of Collagen I mRNA in livers of groups of Bile Duct Ligated (BDL) rats (columns as described for FIG. 1A). The asterisk (*) indicates that the level of collagen I mRNA in livers of vehicle-, GW4064- or TUDCA-treated BDL rats is significantly different (p<0.05) than the level in sham operated rats.

FIG. 1C graphs the level of TGFbeta1 mRNA in livers of groups of Bile Duct Ligated (BDL) rats (columns as described for FIG. 1A). The asterisk (*) indicates that the level of TGFbeta1 mRNA in livers of vehicle-treated BDL rats is significantly different (p<0.05) than levels in sham operated rats. The pound sign (#) indicates that the level of TGFbeta1 mRNA in livers of GW4064-treated BDL rats is significantly different (p<0.05) than levels in vehicle BDL rats.

FIG. 2A graphs the level of Smooth Muscle Actin (SMA) mRNA in isolated rat hepatic stellate cells grown in media containing either 0.1% DMSO (control; white bar) or 10 uM GW4064 with 0.1% DMSO (GW4064; shaded bar). Total RNA was extracted from the cells at days 4, 6 and 8, as indicated. SMA expression is normalized to 18S.

FIG. 2B graphs the level of Collagen mRNA in isolated rat hepatic stellate cells grown in media containing either 0.1% DMSO (control; white bar) or 10 uM GW4064 with 0.1% DMSO (GW4064; shaded bar). Total RNA was extracted from the cells at days 0, 2, 4, 6 and 8, as indicated. Collagen expression is normalized to 18S.

FIG. 2C graphs the level of Fibronectin mRNA in isolated rat hepatic stellate cells grown in media containing either 0.1% DMSO (control; white bar) or 10 uM GW4064 with 0.1% DMSO (GW4064; shaded bar). Total RNA was extracted from the cells at days 2, 4, 6 and 8, as indicated. Fibronectin expression is normalized to 18S.

FIG. 3A graphs the level of TGFbeta1 mRNA (relative quantification) in livers of groups of rats treated with a single dose of carbon tetrachloride as an acute model of liver fibrosis, where the first column is Control rats, the second column is rats treated with a single dose of CCl4 followed by four days dosing with vehicle, and the third column is rats treated with CCl4 followed by four days dosing with the FXR agonist GW4064. The asterisk indicates that the level of TGFbeta1 mRNA in vehicle+CCl4-treated rats is significantly different (p<0.05) than the level in Control rats.

FIG. 3B graphs the level of Smooth Muscle Actin (SMA) mRNA (relative quantification) in livers of groups of rats as described for FIG. 3A. The asterisk (*) indicates that the level of SMA mRNA in livers of vehicle+CCl4-treated rats is significantly different (p<0.05) than the level in Control rats; the pound sign (#) indicates that the level of SMA mRNA in livers of CCl4+GW4064 rats is significantly different (p<0.05) than the level in vehicle+CCl4-treated rats.

FIG. 3C graphs the level of Collagen I mRNA (relative quantification) in livers of groups of rats as described for FIG. 3A. The asterisk (*) indicates that the level of Collagen I mRNA in livers of vehicle+CCl4-treated rats is significantly different (p<0.05) than the level in Control rats; the pound sign (#) indicates that the level of Collagen I mRNA in livers of CCl4+GW4064 rats is significantly different (p<0.05) than the level in vehicle+CCl4-treated rats.

FIG. 4A graphs the level of Collagen mRNA in isolated rat hepatic stellate cells grown in media containing either 0.1% DMSO (vehicle control; white bar) or 3 uM GW4064 with 0.1% DMSO (shaded bar). Total RNA was extracted from the cells at days 0, 2, 4, 6 and 8, as indicated. Collagen expression is normalized to 18S. Asterisk indicates p<0.05 compared to the vehicle control.

FIG. 4B graphs the level of Smooth Muscle Actin (SMA) mRNA in isolated rat hepatic stellate cells grown in media containing either 0.1% DMSO (vehicle control; white bar) or 3 uM GW4064 with 0.1% DMSO (shaded bar). Total RNA was extracted from the cells at days 4, 6 and 8, as indicated. SMA expression is normalized to 18S. Asterisk indicates p<0.05 compared to the vehicle control.

FIG. 4C graphs the level of Fibronectin mRNA in isolated rat hepatic stellate cells grown in media containing either 0.1% DMSO (vehicle control; white bar) or 3 uM GW4064 with 0.1% DMSO (shaded bar). Total RNA was extracted from the cells at days 2, 4, 6 and 8, as indicated. Fibronectin expression is normalized to 18S. Asterisk indicates p<0.05 compared to the vehicle control.

FIG. 5A graphs the level of Smooth Muscle Action (SMA) mRNA in liver tissue from naïve rats (white bar) and rats treated with CCL₄+Vehicle (black bars), CCL₄+GW4064 (striped bars), or CCL₄+Silymarin (stippled bars).

FIG. 5B graphs the level of Collagen mRNA in liver tissue from naïve rats (white bar) and rats treated with CCL₄+Vehicle (black bars), CCL₄+GW4064 (striped bars), or CCL₄+Silymarin (stippled bars). The asterisk (*) indicates p<0.05 compared to the Naïve rats at 8 week timepoint; and the pound sign (#) indicates p<0.05 compared to the CCl₄+Vehicle rats at 8 week timepoint.

FIG. 5C graphs the level of TIMP-1 mRNA in liver tissue from naive rats (white bar) and rats treated with CCL₄+Vehicle (black bars), CCL₄+GW4064 (striped bars), or CCL₄+Silymarin (stippled bars). The asterisk (*) indicates p<0.0⁵ compared to the Naive rats at 8 week timepoint; and the pound sign (#) indicates p<0.05 compared to the CCl₄+Vehicle rats at 8 week timepoint.

FIG. 5D graphs the level of TGFb1 mRNA in liver tissue from naive rats (white bar) and rats treated with CCl₄+Vehicle (black bars), CCL₄+GW4064 (striped bars), or CCL₄+Silymarin (stippled bars). The asterisk (*) indicates p<0.05 compared to the Naïve rats at 8 week timepoint; and the pound sign (#) indicates p<0.05 compared to the CCl₄+Vehicle rats at 8 week timepoint.

FIG. 6 graphs the level of serum TIMP1 in naive rats (white bar) and rats treated with CCL₄+Vehicle (black bars), CCL₄+GW4064 (striped bars), or CCL₄+Silymarin (stippled bars). The asterisk (*) indicates p<0.05 compared to the Naïve rats at 8 week timepoint; and the pound sign (#) indicates p<0.05 compared to the CCl₄+Vehicle rats at 8 week timepoint.

### DETAILED DESCRIPTION OF THE INVENTION

Using a selective FXR agonist, it has now been found in well-characterized models of fibrosis that FXR agonists possess anti-fibrotic properties, and represent a therapeutic approach for the treatment of fibrotic disease.

Fibrosis

Fibrotic disorders can be characterized as acute or chronic, but share the common characteristic of excessive collagen accumulation and an associated loss of function as normal tissue is replaced or displaced by fibrotic tissue. Acute forms of fibrosis include response to trauma, infections, surgery, burns, radiation and chemotherapy. Chronic forms of fibrosis may be due to viral infection, diabetes, obesity, fatty liver, hypertension, scleroderma and other chronic conditions that induce fibrosis.

Organs that are most commonly affected by fibrosis include liver, kidney and lung. Fibrosis may also occur in the heart, and in structures of the eyes. Organ fibrosis can cause the progressive loss of organ function. Retroperitoneal fibrosis (including idiopathic retroperitoneal fibrosis) may not originate from any major organ, but can involve and adversely affect the function of organs such as the kidneys.

Accordingly, as used herein, the term fibrosis refers to all recognized fibrotic disorders, including fibrosis due to pathological conditions or diseases, fibrosis due to physical trauma (‘traumatic fibrosis’), fibrosis due to radiation damage, and fibrosis due to exposure to chemotherapeutics. As used herein, the term “organ fibrosis” includes but is not limited to liver fibrosis, fibrosis of the kidneys, pulmonary fibrosis, cardiac fibrosis, and fibrosis of ocular structures. “Traumatic fibrosis” includes but is not limited to fibrosis secondary to surgery (surgical scarring), accidental physical trauma, burns, and hypertrophic scarring.

As used herein, “liver fibrosis” includes liver fibrosis due to any cause, including but not limited to virally-induced liver fibrosis such as that due to hepatitis B and C; exposure to alcohol (alcoholic liver disease), pharmaceutical compounds, oxidative stress, cancer radiation therapy or industrial chemicals; and diseases such as primary biliary cirrhosis, fatty liver, obesity, non-alcoholic steatohepatitis, cystic fibrosis, hemochromatosis, and auto-immune hepatitis. Current therapy in liver fibrosis is primarily directed at removing the causal agent, e.g., removing excess iron (hemochromatosis), viral load (chronic viral hepatitis), or exposure to toxins (alcoholic liver disease). Anti-inflammatory drugs such as corticosteroids and colchicine are also known for use in treating inflammation that can lead to liver fibrosis. Other strategies for treating liver fibrosis are under development (see, e.g., Murphy et al., Expert Opin. Investig. Drugs 11:1 575 (2002); Bataller and Brenner, Semin. Liver Dis. 21:437 (2001)).

The response of the liver to hepatocellular damage, similar to wound healing in other tissues, includes inflammation and tissue remodeling, with associated changes in the quantity and quality of the extracellular matrix. Progressive accumulation of extracellular matrix proteins, including collagen types I and III, eventually distorts the architecture of the liver by forming a fibrous scar, resulting in disrupted blood flow and an eventual deterioration in hepatic function. Hepatic stellate cells (HSC) have been identified as important mediators of the fibrotic process in the liver, and are believed to be primarily responsible for the synthesis of excess extracellular matrix seen in liver disease. Liver injury can result in quiescent HSCs converting to activated myofibroblast-like cells that proliferate, migrate, recruit inflammatory cells, and synthesize collagens and other extracellular matrix proteins. Various cytokines are reported to activate HSCs, including transforming growth factor B (TGFb). In liver, cholangiocyte production of TGFb is thought to be a key initiating step in the fibrotic process. Following liver injury, HSCs synthesize alpha-smooth muscle actin as part of the migration response, consequently a marked accumulation of alpha-smooth muscle actin (a-SMA) can be seen at areas of active liver fibrogenesis.

As is known in the art, liver fibrosis may be clinically classified into five stages (S0 to S4) of severity, usually based on histological examination of a biopsy specimen. S0 indicates no fibrosis, whereas S4 indicates cirrhosis. While various criteria for staging the severity of liver fibrosis exist, in general early stages of fibrosis are identified by discrete, localized areas of scarring in one portal (zone) of the liver, whereas later stages of fibrosis are identified by bridging fibrosis (scarring that crosses zones of the liver).

FXR Agonists and Fibrosis

The present inventors, in studying the effects of an FXR agonist (GW4064) in cholestasis using bile duct ligated (BDL) rats, noted decreased bile duct proliferation in animals treated with GW4064; bile duct proliferation has been suggested as a marker of fibrosis. This particular study was not long enough in duration to allow the development of fibrotic changes in the liver. Whether the decreased bile duct proliferation was secondary to the decreased bile acid level in the liver or due to additional anti-fibrotic effects of FXR was not immediately apparent.

The present inventors accordingly further investigated TGFb1 mRNA in these BDL rats, and noted that TGFb1 mRNA was reduced in BDL rats receiving GW4064, compared to BDL rats receiving vehicle or taurine-conjugated ursodeoxycholic acid (TUDCA) (measured at four days post bile duct ligation). During BDL, damage to the cholangiocytes lining the bile ducts results in increased release of TGFb. As TGFb is involved in the activation of HSCs and the subsequent collagen and smooth muscle actin (SMA) expression, the present researchers identified the relative decrease in TGFb mRNA in GW4064-treated BDL rats as suggestive of protection by GW4064 against fibrotic changes secondary to cholestasis. In humans, liver fibrosis secondary to long-standing cholestatic disease is known to occur. Experimentally, bile duct ligation in rats as a model of cholestasis is also known to cause liver fibrosis, however, histopathologic evidence of fibrosis would typically not be expected to occur at only four days post bile duct ligation.

In order to determine if the potential anti-fibrotic effects of GW4064 seen in the BDL rat model were due solely to a reduction in the bile acid concentration, or if FXR agonists might have anti-fibrotic activity independent of modulation of bile acid concentration, the present researchers further studied the possible anti-fibrotic effect of the FXR agonist GW4064 using rat hepatic stellate cells in vitro. Activation of hepatic stellate cells into myofibroblasts has been identified as an important step in the development of liver fibrosis; transdifferentiation of hepatic stellate cells is believed to be driven by cytokines, including TGFb. (See e.g., Safadi et al., MedGenMed. 4:27 (2002); Shimizu et al., Curr Drug Targets Infect Disord1:227 (2001), Gressner et al., Front Biosci. 7:793 (2002)).

In the present in vitro studies reported herein, primary rat hepatic stellate cells (HSCs) were found to express FXR. Over an eight day culture period the hepatic stellate cells adopted an “activated” morphology and began to express SMA, collagen, and fibronectin. By the eighth day there was a decrease in all three markers in cells treated with GW4064 (see Example 2). While not wishing to be held to a single theory, the present inventors believe that HSCs contain functional Farnesoid X Receptors, and that activation of these receptors by an FXR agonist compound provides a protective effect against fibrotic changes.

Additional studies (described below) were conducted using the rats treated with carbon tetrachloride to induce liver fibrosis, an accepted animal model of hepatic fibrosis (see, e.g., Wasser and Tan, Ann. Acad. Med. Singapore 28:109 (1999); Tsukamoto et al., Semin. Liver Dis. 10:56 (1990)). The fibrotic changes seen in the carbon tetrachloride (CCl₄) model of liver fibrosis are not due to increased bile acids (compare to bile duct ligation model of cholestasis).

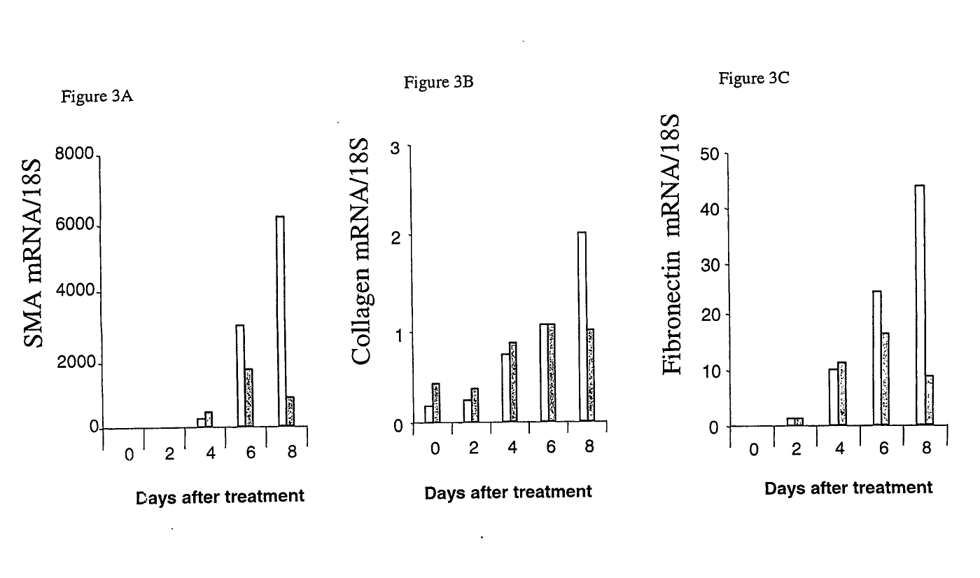

In the first in vivo study (acute model; see Example 3), groups of rats were treated with vehicle (control rats), with CCl₄ and vehicle, or with CCl₄ and the FXR agonist GW4064. After four days of treatment, an increase in TGFb1, SMA and Collagen I mRNA was noted in liver tissue from Group 2 (CCl₄+vehicle) rats, compared to control rats. (See FIG. 3). TGFb1, SMA and Collagen I mRNA measurements were not significantly increased in rats treated with GW4064 compared to control rats. Additionally, the differences in SMA and Collagen mRNA levels were significantly decreased in GW4064-treated rats compared to rats treated with CCl₄ and vehicle.

In the second in vivo study (chronic model; see Example 4), groups of rats received twice weekly intraperitoneal injections of CCl₄ for up to eight weeks. After two weeks, groups of rats additionally were administered twice daily vehicle, 30 mg/kg GW4064 in vehicle, or silymarin in vehicle. Histopathological examination revealed that livers from rats receiving CCl₄+vehicle had increased collagen deposition, and by 8 weeks were cirrhotic. The livers of rats receiving CCl₄, followed by treatment with GW4064 or silymarin had reduced collagen deposition compared to the livers from the CCl₄+vehicle animals at six and eight weeks. In liver tissue, significant reductions in Collagen I and TIMP1 (tissue inhibitor of metalloproteinase) mRNA were seen in rats treated with CCl₄+GW4064, compared to CCl₄+vehicle treated rats. Additionally, significant reduction in serum TIMP1 was seen in CCl₄+GW4064 treated rats, compared to CCl₄+vehicle treated rats.

Accordingly, data from these experiments indicate that FXR agonists have therapeutic utility in the prevention or treatment of organ fibrosis, and in particular have therapeutic utility in the prevention or treatment of liver fibrosis.

Yu et al. reported increased carbon tetracholoride-induced liver injury and fibrosis in mice deficient in the cell surface tyrosine kinase receptor Fibroblast Growth Factor Receptor 4 (FGFR4) (Am. J. Pathology, 161:2003 (2002)). Fibroblast Growth Factor 19 (FGF19) is a high affinity ligand for FGFR4 (Xie et al., Cytokine 11:729 (1999)). As reported in PCT/US03/08634, filed March 2003 in the name of SmithKline Beecham Corporation, expression of Fibroblast Growth Factor 19 (FGF19) was increased in human hepatocytes treated with the FXR agonist compound GW4064 compared to control cells. See also Holt et al., Genes & Develop. 17:1581 (2003), reporting that FXR directly regulates expression of FGF19. While not wishing to be held to a single theory, the present inventors propose that administration of an FXR agonist may protect against fibrosis in the liver due to an increase in FGF19 expression, and subsequent effects on the the FGF19-FGFR4 pathway in liver cells; and propose that this mechanism is relevant in kidney, lung and other organ fibrosis as well

Ligand binding of the FXR nuclear receptor can result in the alteration of expression of various genes that FXR aids in regulating, including genes involved in lipid absorption and the reabsorption of bile acids in small intestine and lipid homeostasis in the liver. Examples of such genes include, but are not limited to, genes involved in bile acid transport, lipid absorption, cholesterol biosynthesis, proteolysis, amino acid metabolism, glucose biosynthesis, protein translation, electron transport, and hepatic fatty acid metabolism. FXR often functions as a heterodimer with the Retinoid X Receptor (the FXR/RXR heterodimer).

“FXR agonist” as used herein refers to an agent that directly binds to and upregulates the activity of FXR. In the present methods, preferred FXR agonists are small molecule organic compounds, preferably synthetic small molecule organic compounds, and may be non-steroidal synthetic small molecule organic compounds. Preferred FXR agonist compounds for use in the present methods do not include naturally occurring bile acids. However, in one embodiment it is contemplated that the methods of the present invention utilize synthetic FXR agonists in combination with a naturally occurring non-toxic bile acid, such as ursodeoxycholic acid, as an aid in preventing possible depletion of fat-soluble vitamins secondary to treatment with an FXR agonist. Accordingly, synthetic FXR agonists may be administered concurrently with the naturally occurring non-toxic bile acid, either as separate entities or as a single formulation comprising both synthetic FXR agonist and naturally occurring bile acid.

As used herein, the term “small molecule organic compound” refers to a chemical compound that is an organic compound having a molecular weight less than about 10,000 daltons, and more preferably having a molecular weight less than about 7,500 daltons, less than about 5,000 daltons, or less than about 2,500 daltons. As used herein, the term “synthetic compound” refers to a chemical compound where the compound structure is not known to occur in nature, whereas the term “naturally-occurring compound” refers to a chemical compound isolated from or known to occur in natural sources, such as cells, plants, fungi, animals and the like.

The bile acids chenodeoxycholic acid (CDCA), deoxycholic acid (DCA), and the taurine and glycine conjugates thereof selectively activate FXR (WO 0037077, Glaxo Group Limited). As used herein, the term FXR agonist” refers to compounds that achieve at least about 25% activation of FXR relative to CDCA, the positive control in the assay methods described in PCT Publication No. WO 00/37077 published 29 Jun. 2000 to Glaxo Group Limited, the subject matter of which is incorporated herein by reference in its entirety. Preferably, the compounds used in the methods of this invention achieve at least about 50% activation of FXR in the scintillation proximity assay or the HTRF assay as described in PCT Publication No. WO 00/37077; more preferably, the compounds achieve at least about 75%, 80%, 90%, 95%, 97% or greater activation of FXR in the scintillation proximity assay or the HTRF assay as described in PCT Publication No. WO 00/37077.

An FXR agonist for use in the present invention is the compound known as GW4064, as disclosed in PCT Publication No. WO 00/37077 published 29 Jun. 2000 to Glaxo Group Limited, which describes FXR ligand compounds characterized by the following formula (I) 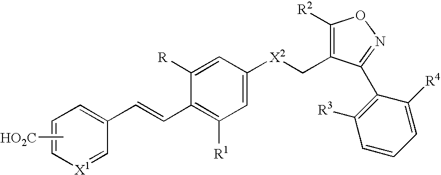
wherein X¹ is CH or N; X² is O or NH; R and R¹ may independently be H, lower alkyl, halogen, or CF₃; R² is lower alkyl; R³ and R⁴ may independently be H, lower alkyl, halogen, CF₃, OH, O-alkyl, or O-polyhaloalkyl.

GW4064, an example of a compound of Formula (I), is a potent and selective FXR ligand and has the following formula (II):

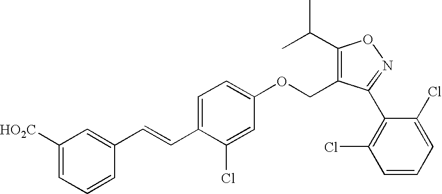

FXR agonists for use in the present invention may further include 6α-alkyl-substituted analogues of chenodeoxycholic acid (CDCA), such as 6α-ethyl-chenodeoxycholic acid (6-ECDCA) (Pellicciari et al., J. Med. Chem., 45:3569 (2002); WO 02/072598 published 19 Sep. 2002 in the name of Pellicciari), and 3-deoxychenodeoxycholic acid.

Additional FXR ligands useful in the present inventions can be identified routinely by those of skill in the art based upon assays described in PCT/US99/30947, the teachings of which are herein incorporated by reference in their entirety. In a preferred embodiment, FXR ligands are identified using a nuclear receptor-peptide assay for identifying ligands. This assay utilizes fluorescence resonance energy transfer (FRET) and can be used to test whether putative ligands bind to FXR. The FRET assay is based upon the principle that ligands induce conformational changes in nuclear receptors that facilitate interactions with coactivator proteins required for transcriptional activation. In FRET, a fluorescent donor molecule transfers energy via a non-radioactive dipole-dipole interaction to an acceptor molecule (which is usually a fluorescent molecule). FRET is a standard spectroscopic technique for measuring distances in the 10-70 Å range. Upon energy transfer, which depends on the R⁻⁶ distance between the donor and acceptor, the donor's fluorescence is reduced, and the acceptor fluorescence is increased, or sensitized. FRET is frequently used in both polymer science and structural biology and has recently been used to study macromolecular complexes of DNA, RNA, and proteins. In addition, Mathis has used europium cryptates with the multichromophoric Allophycocanin to achieve an extremely large R₀ of 90 Å (Mathis et al. Clin. Chem. 1993 39:1953-1959).

The compounds of Formula (I) or (II) can be synthesized using standard techniques of organic chemistry. A convergent strategy can be employed in which a hydroxystilbene and a hydroxymethyisoxazole are prepared independently and then condensed using a Mitsunobu coupling to generate the ether linkage. Compounds with anilino linkages can be prepared by converting the hydroxyl residue of a hydroxymethyisoxazole into a leaving group, such as bromide or mesylate, followed by reaction with an aminostilbene.

Hydroxymethylsoxazoles can be prepared by the condensation of a beta-keto ester enolate with an α-halo-substituted hydroxamic acid. The resulting esters can be reduced to an alcohol with a metal hydride reducting agent such as diisobutyl aluminum hydride (DIBAL).

Hydroxystilbenes can be prepared by Horner-Wadsworth-Emmons coupling of an aryl aldehyde and an arylmethylene phosphonate ester, or by Heck coupling of a styrene with an arylbromide, iodide, or triflate in the presence of a palladium catalyst. Using standard chemical methods, tritium or iodine 125 can be incorporated into the compounds of formula (1) and (II).

In one embodiment, formula I (GW4064) is synthesized in accordance with procedures described by Maloney et al. J. Med. Chem. 43:2971-4.

### DEFINITIONS

The methods of the present invention are directed to the use of FXR agonists in methods of treating fibrosis, including organ fibrosis and liver fibrosis.

“Treating fibrosis” or ‘treatment of fibrosis’, as used herein, includes both prophylactic and therapeutic treatment: methods that prevent or reduce the manifestations of fibrosis in a manner beneficial to the health or physical well-being of the individual, such as to reduce symptoms or disease markers or to prevent, slow, halt or reduce one or more molecular, macromolecular or cellular mechanisms of fibrosis.

As used herein, ‘prophylactic treatment’ refers to the treatment of a subject with a condition known to result in fibrosis or otherwise at increased risk of fibrosis, but who does not yet have histological evidence of fibrosis (e.g., in the case of liver fibrosis, an individual who would be designated as S0), in order to prevent fibrosis or reduce the extent of fibrotic changes that would be expected to occur in the absence of treatment.

As used herein, ‘therapeutic treatment’ refers to treatment of a subject with histopathological fibrotic changes (e.g., in the case of liver fibrosis, an individual who would be designated as S1-S4) or changes in disease markers consistent with fibrotic disease in the absence of biopsy. Therapeutic treatment is designed to prevent, or decrease the rate of, further fibrotic changes that would be expected to occur in the absence of treatment.

Preferred subjects for the methods of the present invention are mammals, including but not limited to humans, canines and felines.

As used herein, a ‘therapeutically effective amount’ of an FXR agonist, in the treatment of fibrotic disease, indicates an amount of FXR agonist that prevents or reduces fibrotic changes, or slows the rate of fibrotic changes, as compared to the fibrotic changes that would occur in the absence of treatment. A reduction in or slowing of fibrotic changes may be measured or ascertained using any suitable means as are known in the art. Biopsy with histological examination remains a recognized method of assessing fibrotic changes in an organ. Additionally, serum markers or other biochemical markers of fibrosis may be utilized to assess the degree of fibrosis, e.g., in subjects with liver fibrosis due to viral infection (see, e.g., Afdahl, Hepatology 37:972 (2003); Bonancini et al., Am J Gastroenterol. 92:1302 (1997); Rossi et al., Clin Chem. 9:450 (2003)).

Formulations, Pharmaceutical Compositions

FXR agonists used in the methods of the present invention are conveniently administered in the form of pharmaceutical compositions. Such pharmaceutical compositions comprising a FXR agonist may conveniently be presented for use in a conventional manner in admixture with one or more physiologically acceptable carriers or excipients.

FXR agonists useful in the methods of the present invention may be formulated for administration in any suitable manner. They may, for example, be formulated for topical administration or administration by inhalation or, more preferably, for oral, transdermal or parenteral administration. The pharmaceutical composition may be in a form such that it can effect controlled release of the FXR agonist. A particularly preferred method of administration, and corresponding formulation, is oral administration. For oral administration, the pharmaceutical composition may take the form of, and be administered as, for example, tablets (including sub-lingual tablets) and capsules (each including timed release and sustained release formulations), pills, powders, granules, elixirs, tinctures, emulsions, solutions, syrups or suspensions prepared by conventional means with acceptable excipients.

For oral administration in the form of a tablet or capsule, the active FXR agonist can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. Oral fluids such as solution, syrups and elixirs can be prepared in dosage unit form so that a given quantity contains a predetermined amount of the compound. Where appropriate, dosage unit formulations for oral administration can be microencapsulated. The formulation can also be prepared to prolong or sustain the release as for example by coating or embedding particulate material in polymers, wax or the like.

FXR agonists for use in the methods of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

FXR agonists for use in the methods of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

The present invention includes pharmaceutical compositions containing 0.1 to 99.5%, more particularly, 0.5 to 90% of an FXR agonist in combination with a pharmaceutically acceptable carrier.

Compositions comprising a FXR agonist may also be administered in nasal, ophthalmic, otic, rectal, topical, intravenous (both bolus and infusion), intraperitoneal, intraarticular, subcutaneous or intramuscular inhalation or insufflation form, all using forms well known to those of ordinary skill in the pharmaceutical arts. For transdermal administration, the pharmaceutical composition comprising the FXR agonist may be given in the form of a transdermal patch, such as a transdermal iontophoretic patch.

For parenteral administration, the pharmaceutical composition comprising the FXR agonist may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilizing and/or dispersing agents. For administration by injection these may take the form of a unit dose presentation or as a multidose presentation preferably with an added preservative. Alternatively for parenteral administration the active ingredient may be in powder form for reconstitution with a suitable vehicle.

Pharmaceutical compositions comprising a FXR agonist are administered in an amount effective for treatment or prophylaxis of fibrotic diseases, and fibrosis resulting from such diseases or from injuries. Initial dosing in human is accompanied by clinical monitoring of symptoms for such conditions. For administration particularly to mammals, and particularly humans, it is expected that the daily dosage level of the active agent will be from 0.1 mg/kg to 100 mg/kg and typically around 30 mg/kg. It will be appreciated that optimum dosage will be determined by standard methods for each treatment modality and indication, taking into account the indication, its severity, route of administration, complicating conditions and the like. The physician in any event will determine the actual dosage that will be most suitable for an individual and will vary with the age, weight and response of the particular individual. The effectiveness of a selected actual dose can readily be determined, for example, by measuring clinical symptoms or standard indicia of fibrosis or fibrotic changes after administration of the selected dose. The above dosages are exemplary and there can, of course, be individual instances where higher or lower dosage ranges are merited, and such are within the scope of this invention. For conditions or disease states as are treated by the present invention, maintaining consistent daily levels in a subject over an extended period of time, e.g., in a maintenance regime, can be particularly beneficial.

The following nonlimiting examples are provided to further illustrate the present invention.

### EXAMPLE 1

### Bile Duct Ligated Rats

A study of the effects of an FXR agonist (GW4064) on the livers of bile duct ligated (BDL) rats was carried out. This study was initially devised to examine the effects of FXR agonist on non-fibrotic liver damage due to cholestasis. The present researchers additionally went on to examine samples from these experiments for markers of liver fibrosis.

Male Sprague-Dawley rats (approximately 300 grams) were obtained from Charles River Laboratories Inc (Raleigh, N.C.) and were maintained on a 12 hour light/12 hour dark light cycle. Animals were anesthetized by the administration of 2-3% isoflurane. Laparotomy was performed under sterile technique and the liver and duodenum gently displaced to reveal the common bile duct. The bile duct was separated from the surrounding tissue and two ligatures of 4-0 Ethilon were placed around it. The bile duct was clamped between the two ligatures with an aneurysm clamp and the ligatures drawn tight. An additional ligature was placed proximal to the first (near the liver). The clamp was removed and the bile duct severed between the ligatures. The muscle wall was closed with 4-0 Vicryl and the skin closed with staples. Sham controls underwent laparotomy, but the bile ducts were not ligated or transected. Animals were allowed food and water ad libitum throughout the study period. Animals were anesthetized with 2-3% isoflurane and sacrificed by cardiac puncture.

Twenty-four hours after surgery, groups of rats (n=6-8) received intraperitoneal injections once daily for four days, of either (a) 5 ml/kg corn oil (vehicle); (b) 30 mg/kg GW4064 in corn oil; or (c) 15 mg/kg TUDCA in corn oil (taurine-conjugated ursodeoxycholic acid, used clinically to treat cholestasis). Sham operated animals received intraperitoneal injections of 5 ml/kg corn oil vehicle 24 hours after sham operation. Four hours after the last dose on day four, blood and livers were collected.

Serum activities of alanine aminotransferase (ALT), asparate aminotransferase (AST), sorbital dehydrogenase (SDH), y-glutamyl transferase (GGT), lactate dehydrogenase (LDH), alkaline phosphatase (ALP) and total bilirubin were determined. Serum bile acids (BILEA) were determined using a commercially available assay (Sigma Chemical Co., St Louis, Mo.).

Serum levels of ALT, AST, SDH, ALP and GGT increased in response to BDL, while LDH activity decreased. GW4064 treatment resulted in reductions in serum levels of ALT, AST, and LDH in BDL rats, compared to vehicle-treated BDL rats. GW4064 treatment did not significantly reduce serum levels of ALP, SDH, GGT, bile acids or total bilirubin, compared to vehicle treated BDL rats. TUDCA treated BDL rats showed a significant decrease only in LDH, compared to vehicle treated BDL rats. (Data not shown).

Liver samples from the BDL animals were examined histologically for necrosis and bile duct proliferation (results not shown). Increased levels of bile duct proliferation were found in the vehicle-treated group; these animals also showed hepatic parenchymal necrosis with inflammatory cell infiltration. In comparison, sections from the GW4064-treated BDL rats had qualitatively fewer and smaller necrotic lesions and decreased fatty cell degeneration. GW4064 treated BDL rats also showed reduced bile duct proliferation. The number of mitotic nuclei was also reduced by GW4064 treatment compared to vehicle treatment. Sections from TUDCA treated BDL rats did not appear to differ substantially from vehicle treated BDL rats.

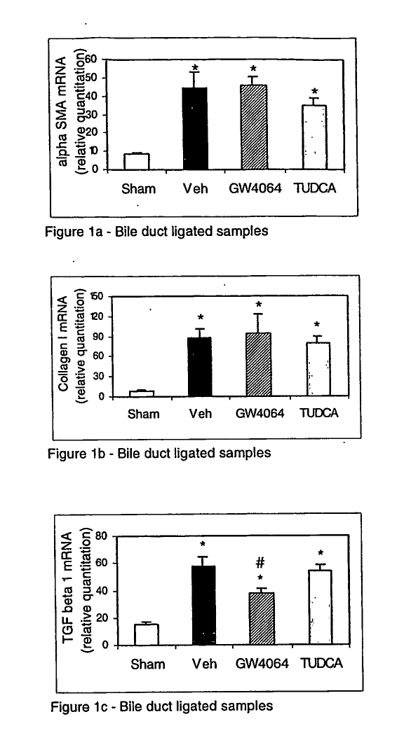

In humans, liver fibrosis secondary to cholestatic disease is known to occur clinically. Experimentally, bile duct ligation in rats as a model of cholestasis is also known to cause liver fibrosis. In view of this, the present researchers additionally examined the BDL rat liver tissue for markers of liver fibrosis. Levels of Smooth Muscle Actin (SMA) mRNA, Collagen I mRNA, and TGFbeta1 mRNA were measured; results are graphed in FIGS. 1a-1c where white bars=sham operated rats; black bars=vehicle treated BDL rats; striped bars=GW4064 treated BDL rats; and dotted bars=TUDCA treated BDL rats.

As shown in FIGS. 1a-1b, in these rats at day four after bile duct ligation, alpha SMA mRNA and Collagen I mRNA were increased in all three treatment groups of BDL rats, compared to sham operated rats. Levels of TGFb1 mRNA (FIG. 1c) were increased in all BDL rats compared to sham-operated rats, but it was noted that TGFb1 mRNA was significantly reduced in BDL rats receiving GW4064, compared to BDL rats receiving vehicle or TUDCA.

As TGFb is involved in the control of collagen and SMA expression, the relative decrease in TGFb mRNA in GW4064-treated BDL rats at four days post surgery was suggestive of an early sign of possible protection by GW4064 against fibrotic injury secondary to cholestasis.

### EXAMPLE 2

### In Vitro Stellate Cell Activation

Rat hepatic stellate cells were isolated from male CRL:CD(SD)IGS® rats (Charles River Laboratories) as described previously. Cells were plated into six well plates at a density of 1×10⁶ cells/well in DMEM-F12 media with 20% FCS. Twenty-four hours after plating, the media was replaced with media containing 0.1% DMSO (vehicle control) or 10 uM GW4064 with 0.1% DMSO. Following 2, 4, 6 or 8 days of treatment, total RNA was extracted from the cells using TRIzol reagent (Invitrogen) according to the manufacturer's directions. The RNA was treated with DNase I (Ambion, Austin, Tex.) at 37° C. for 30 min, followed by inactivation at 75° C. for 5 min. RNA was then quantitated using the RiboGreen RNA quantitation kit (Molecular Probes, Eugene, Oreg.). RNA expression was measured by RTQ-PCR using an ABI PRISM 7700 or 7900 Sequence Detection System (PE Applied Biosystems, Foster City, Calif.). Sequences of the gene specific primers and probes used for RTQ-PCR are listed in Table 1. For analysis, gene expression was normalized to 18S RNA.

TABLE 1
Primer-Probe sets and gene abbreviations

| | | |
|---|---|---|
| Forward Primer | Reverse Primer | Probe Sequence* |

| | | | |
|---|---|---|---|
| TGF-B1 | GCTGCTGACCCCCA | GCCACTGCCGGACA | CGCCTGAGTGGCTG |
| Tgfb1(UniGene) | CTGAT | ACTC | TCTTTTGACGT |
| NM021578 (GenBank) | (SEQ ID NO:1) | (SEQ ID NO:2) | (SEQ ID NO:3) |
| SMA | TCCTGACCCTGAAG | GGTGCCAGATCTTT | AACACGGCATCATC |
| Acta2 (UniGene) | TATCCGATA | TCCATGTC | ACCAACTGGGA |
| NM012893 (GenBank) | (SEQ ID NO:4) | (SEQ ID NO:5) | (SEQ ID NO:6) |
| Collagen I | TTCACCTACAGCAC | GATGACTGTCTTGC | ATGGCTGCACGAGT |
| COLIA1 (UniGene) | GCTTGTG | CCCAAGTT | CACACCG |
| Z78279 (GenBank) | (SEQ ID NO:7) | (SEQ ID NO:8) | (SEQ ID NO:9) |
| Fibronectin | CCAAAGCCACCGGA | GCTTGAAGCCAATC | CTCTGCGCTCCATTC |
| Fn1 (Unigene) | GTCTT | CTTGGA | CACCTTATAACACC |
| X15906 (GenBank) | (SEQ ID NO:10) | (SEQ ID NO:11) | (SEQ ID NO:12) |
| TIMP1 | GAACCGCAGCGAGG | GGCAGTGATGTGCA | TCATCGCGGGCCGT |
| Timp1 (UniGene) | AGTTT | AATTTCC | TTAAGGAA |
| U06179 (GenBank) | (SEQ ID NO:13) | (SEQ ID NO:14) | (SEQ ID NO:15) |

*all probes used as FAM -sequence - TAMRA

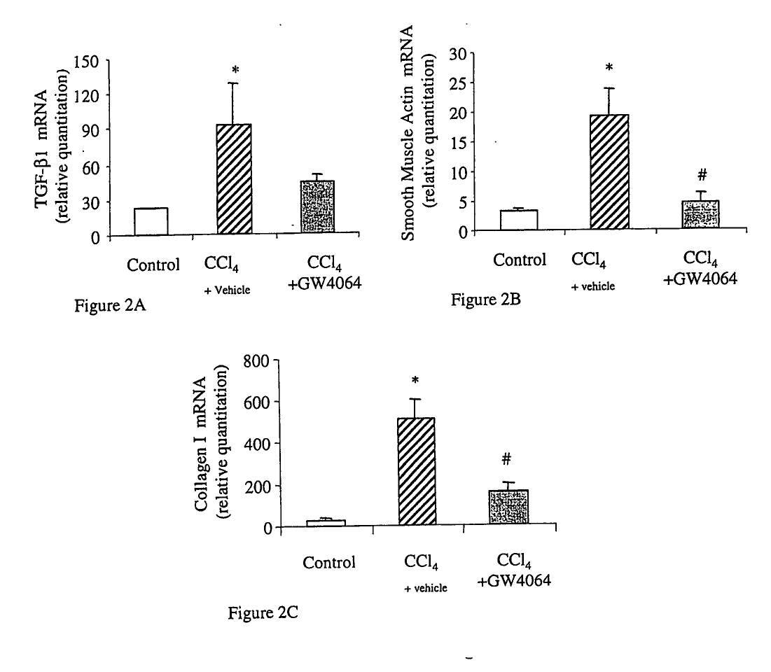

Over the eight day culture period the cells adopted an “activated” morphology and began to express SMA, collagen, and fibronectin As shown in FIGS. 2A-2C, on the eighth day all three markers in the GW4064 group were decreased compared to the control group. SMA was decreased by 84%, collagen by 51% and fibronectin by 79%. SMA and fibronectin were also decreased in the GW4064 treatment group at six days (compared to the vehicle control group).

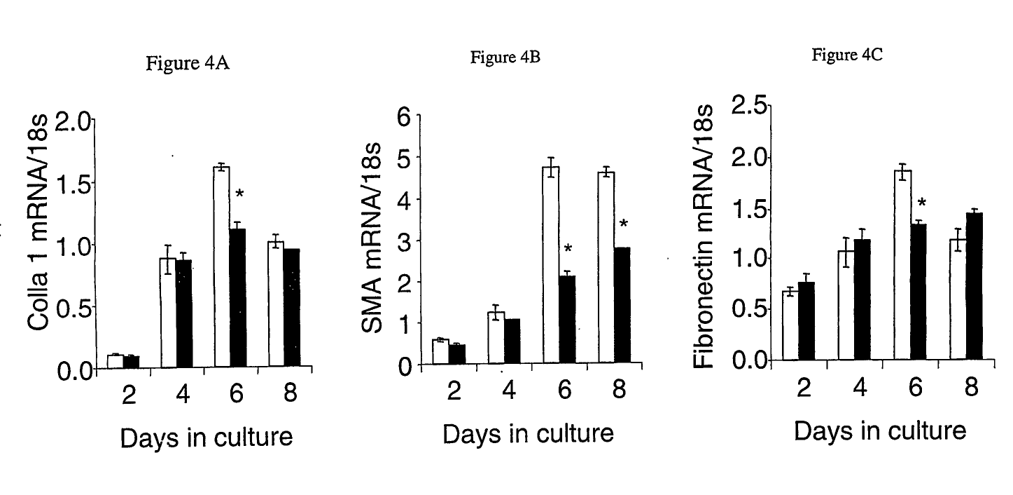

Follow-up studies were conducted using the same study design, except for a lower dose of GW4064 (3 uM). In the vehicle control group, SMA, collagen and fibronectin peaked at six days of culture; collagen and fibronectin expression began to decline following eight days in culture (FIGS. 4A-4C, white bars). In response to GW4064 treatment, at six days SMA was decreased by 55%, collagen by 31% and fibronectin by 28%, whereas at eight days SMA was decreased by 40% but collagen and fibronectin were not significantly different from vehicle. (FIGS. 4a-4c, shaded bars; asterisk indicates p<0.05 compared to the vehicle treatment at the indicated timepoint).

### EXAMPLE 3

### Acute Liver Fibrosis In Vivo Model

Male CRL:CD(SD)IGS® rats (Charles River Laboratories) weighing 250-280 g were randomized into three groups of 5-6 rats per group. On day 1, rats in Group 1 (Control) were given a single intraperitoneal (ip) injection of corn oil (5 ml/kg). Rats in Groups 2 and 3 were given a single ip injection of 30% carbon tetrachloride (CCl₄) in corn oil (5 ml/kg). Beginning four hours after the initial ip injections and continuing for four days, rats in Group 1 (Control) and Group 2 (CCl₄) were given vehicle (20% Encapsin; Cerestar Inc., Ind.) by oral gavage twice daily. Beginning four hours after the initial ip injections and continuing for four days, rats in Group 3 (CCl₄+GW4064) were given 30 mg/kg GW4064 in vehicle by oral gavage, twice daily for four days.

Four hours after the final oral dose on day four, rats were sacrificed under deep anesthesia and blood and livers were collected. A portion of liver from each rat was fixed in 10% phosphate-buffered formalin (pH 7.4). The remaining livers were snap frozen in liquid nitrogen and stored at −80° C. prior to RNA extraction.

Histopathology: The tissues were processed by standard histological techniques. Liver sections were stained with hematoxylin and eosin (H&E) using standard protocols and examined by light microscopy for necrosis and other structural changes. Immunohistochemistry was used to visualize a-smooth muscle actin (DAKO Corporation, Calif.) according to the manufacturer's instructions. Sirius Red (Sigma, St Louis, Mo.) was used to visualize collagen according to the manufacturer's instructions.

Reverse Transcription Quantitative Polymerase Chain Reaction (RTQ-PCR). Total RNA was extracted from rat liver using TRIzol reagent (Invitrogen) according to the manufacturer's directions. The RNA was treated with DNase I (Ambion, Austin, Tex.) at 37° C. for 30 min, followed by inactivation at 75° C. for 5 min. RNA was then quantitated using the RiboGreen RNA quantitation kit (Molecular Probes, Eugene, Oreg.). RNA expression was measured by RTQ-PCR using an ABI PRISM 7700 or 7900 Sequence Detection System (PE Applied Biosystems, Foster City, Calif.). Sequences of the gene specific primers and probes used for RTQ-PCR are listed in Table 1.

Statistical Analysis: All data were analyzed by one-way analysis of variance (ANOVA) followed by Duncan's multiple range test. The 0.05 level of probability was used as the criteria of significance.

Histopathology Results—Collagen: Animals that received corn oil and Encapsin vehicle (controls) had normal collagen staining as visualized with Sirius Red stain (results not shown). In general, collagen was detected only in the pericentral region of the livers of these animals (results not shown). The rats that received the CCl₄ and Encapsi had greatly increased collagen deposition, including areas of bridging fibrosis. Although the livers of rats that received CCl₄ followed by GW4064 in Encapsin had more collagen deposition than the normal livers, the CCl₄+GW4064 livers showed reduced collagen deposition compared to the livers from the CCl₄+V animals (results not shown).

Immunocytochemical Results—SMA: Animals that received corn oil and Encapsin vehicle (controls) had normal SMA as visualized immunohistochemically. The rats that received the CCl₄ and Encapsin had greatly increased SMA deposition (results not shown). Although the livers of rats that received CCl₄ followed by GW4064 in Encapsin had more SMA than the normal livers, the CCl₄+GW4064 livers showed reduced SMA compared to the livers from the CCl_{4b}+V animals (results not shown).

RTQ-PCR Results: A marked induction of TGFb1, SMA and Collagen I mRNA was noted in liver tissue from Group 2 (CCl4) rats, compared to Group 1 (Control) rats. FIGS. 3a-3c where the asterisk (*) indicates p<0.05 compared to Group 1 (Control); and the pound sign (#) indicates p<0.05 compared to Group 2 (CCl4+vehicle) ratsSMA and Collagen I mRNA levels were significantly decreased in group 3 (CCl4+GW4064) compared to group 2 (CCl4+vehicle). SMA, Collagen I and TGFb1 mRNA levels were reduced in the group 3 (CCl4+GW4064) rats to a point where they were not statistically different from control.

### EXAMPLE 4

### Chronic Liver Fibrosis In Vivo Model

Male CRL:CD(SD)IGS® rats (Charles River Laboratories) weighing 250-350 were randomized into 3 groups. All rats received twice weekly intraperitoneal (ip) injections of 20% carbon tetrachloride (CCl₄) in corn oil (5 ml/kg) for the duration of the study. Two weeks after the initiation of the CCl₄ injections, rats in Group 1 (CCL₄+Vehicle) began receiving vehicle (6% Encapsin; Cerestar Inc., Ind.) twice daily, rats in Group 2 (CCL₄+GW4064) began receiving 30 mg/kg GW4064 in vehicle twice daily, and rats in Group 3 (CCL₄+Silyinarin) began receiving silymarin in vehicle twice daily. (Silymarin reduces TGFb1 and collagen deposition in fibrotic rat liver (Jia et al., J Hepatol 2001 September; 35(3):392-8).

Multiple rats from each group were sacrificed after each of four, six and eight weeks on study. Rats were also sacrificed following the two weeks of CCL₄ exposure, prior to initiation of drug treatment, as well as five untreated Naive rats. An additional five untreated Naïve rats were also sacrificed at the eight week timepoint. Plasma, serum, kidneys and livers were collected for analysis. Histopathological, mRNA and serum markers of liver damage and liver fibrosis were examined.

Histopathology: The tissues were processed by standard histological techniques. Liver sections were stained with hematoxylin and eosin (H&E) using standard protocols and examined by light microscopy for necrosis and other structural changes. Immunohistochemistry was used to visualize a-smooth muscle actin (Sigma, St Louis, Mo.) according to the manufacturer's instructions. Sirius Red (Sigma, St Louis, Mo.) was used to visualize collagen according to the manufacturer's instructions. Liver sections were stained with Masson's trichrome (Sigma, St Louis, Mo.) according to the manufacturer's instructions.

Reverse Transcription Quantitative Polymerase Chain Reaction (RTQ-PCR). Total RNA was extracted from sections of rat liver using TRIzol reagent (Invitrogen) according to the manufacturer's directions. The RNA was treated with DNase I (Ambion, Austin, Tex.) at 37° C. for 30 min, followed by inactivation at 75° C. for 5 min. RNA was then quantitated using the RiboGreen RNA quantitation kit (Molecular Probes, Eugene, Oreg.). RNA expression was measured by RTQ-PCR using an ABI PRISM 7700 or 7900 Sequence Detection System (PE Applied Biosystems, Foster City, Calif.). Sequences of the gene specific primers and probes used for RTQ-PCR are listed in Table 1.

Serum Analysis: Serum TIMP1 (R&D Systems, Minneapolis, Minn.) levels were measured according to the manufacturer's directions.

Statistical Analysis: All data were analyzed by one-way analysis of variance (ANOVA) followed by Duncan's multiple range test. The 0.05 level of probability was used as the criteria of significance.

Histopathology Results—Collagen: Naive animals that received no treatment (Naïve) had normal collagen staining as visualized with Sirius Red stain (results not shown). In general, collagen was detected only in the pericentral region of the livers of these animals (results not shown). The rats that received the CCl₄+Vehicle had increased collagen deposition over time. Areas of bridging fibrosis were rare at 2 weeks, common at 4 weeks and prominent at 6 weeks. By 8 weeks the livers were cirrhotic with isolated parenchymal nodules (results not shown). The livers of rats that received CCl₄ followed by GW4064 in Encapsin or Silymarin in Encapsin had reduced collagen deposition compared to the livers from the CCl₄+V animals at the six and eight week timepoints (results not shown).

Masson's Trichrome Results—not completed at this time.

SMA Results: not completed at this time.

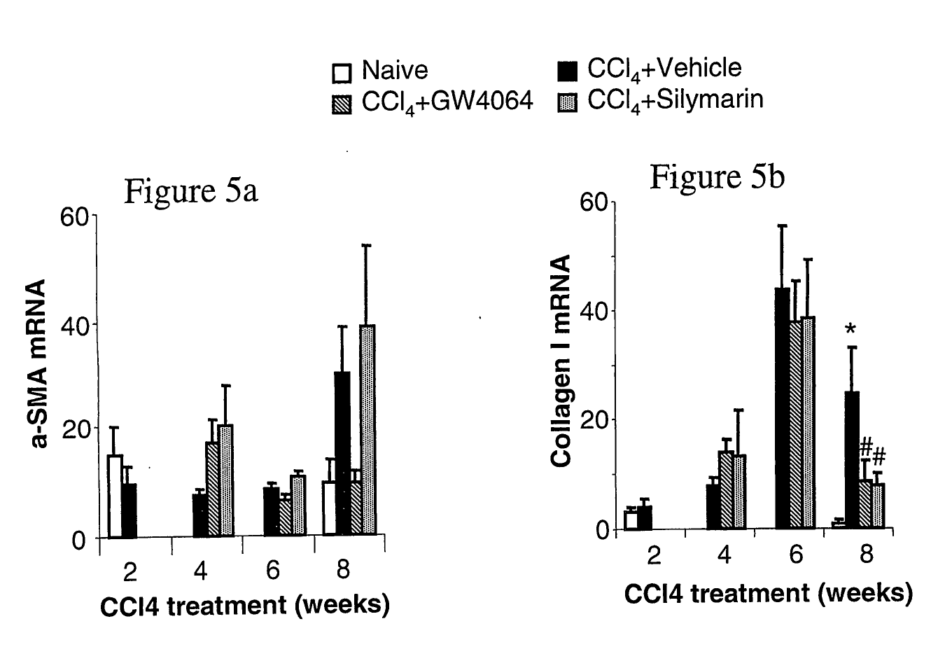

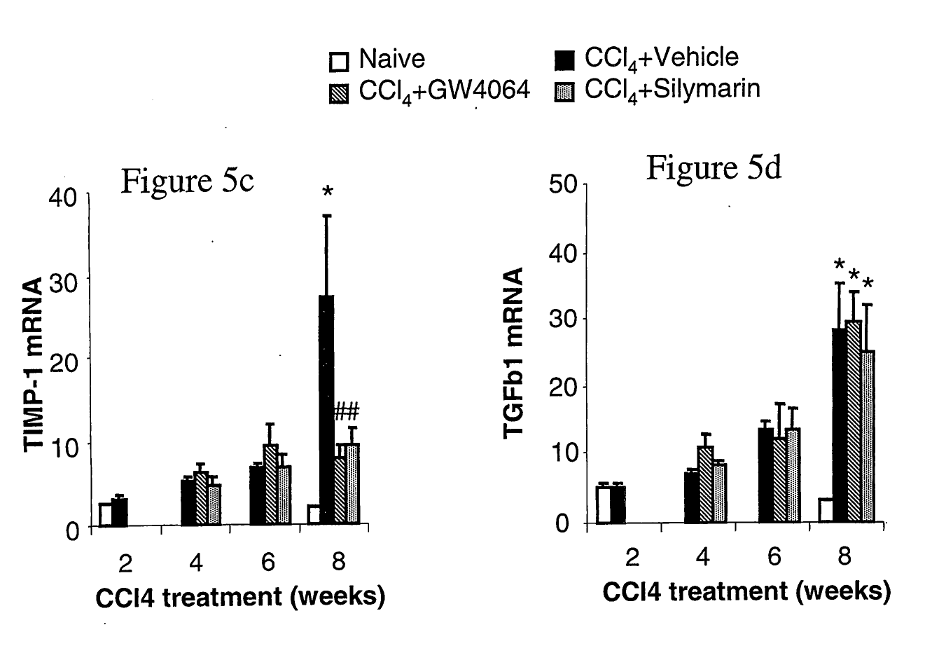

RTQ-PCR Results: Shown in FIGS. 5a-5d, where the asterisk (*) indicates p<0.05 compared to the Naïve rats at 8 week timepoint; and the pound sign (#) indicates p<0.05 compared to the CCl₄+Vehicle rats at 8 week timepoint. A gradual induction of TGFb1, SMA, Collagen I and TIMP1 mRNA was seen in liver tissue from CCl₄+Vehicle rats (solid bars) compared to Naive rats (white bars) over the eight week study period. Statistically significant increases in TGFb1 and TIMP1 mRNA were seen in the CCl₄+Vehicle group compared to Naive only at eight weeks, whereas Collagen I was significantly increased at both 6 and 8-weeks. The induction of SMA mRNA in CCl₄+Vehicle rats did not reach statistical significance compared to Naive.

The liver tissue from CCl₄+GW4064 treated rats (striped bars) had significant reductions in Collagen I and TIMP1 mRNA compared to CCl₄+Vehicle rats (solid bars), although the reduction in SMA was not statistically significant. The liver tissue from CCl₄+silymarin treated rats (stippled bars) had significant reductions in Collagen I and TIMP1 mRNA compared to CCl₄+Vehicle, but no reduction in SMA. TGFb1 mRNA was unchanged by either GW4064 or silymarin treatment.

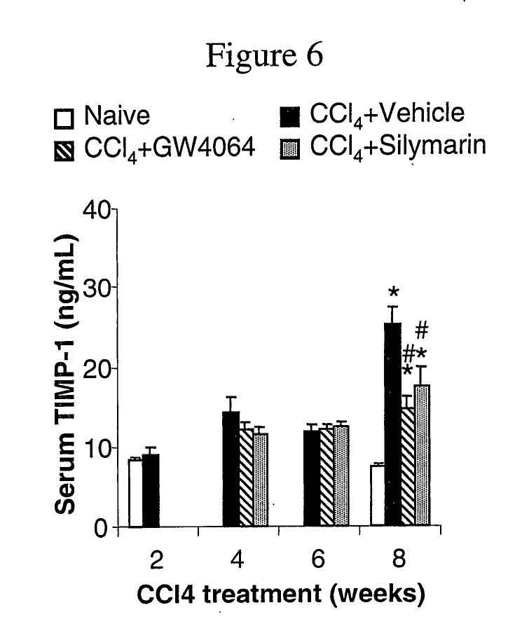

Serum Analysis Results: Results are shown in FIG. 6, where the asterisk (*) indicates p<0.05 compared to the Naïve rats (white bars) at 8 weeks; and the pound sign (#) indicates p<0.05 compared to the CCl₄+Vehicle rats (black bars) at 8 weeks. Results show an induction of serum TIMP1 in CCl₄+Vehicle rats compared to Naïve rats at the eight week timepoint. The serum from CCl₄+GW4064 treated rats (striped bars) and CCl₄+Silymarin treated rats (stippled bars) had significant reductions in serum TIMP1 compared to CCl₄+Vehicle treated rats.

What is claimed is:

1. A method for treating liver fibrosis in a mammalian subject comprising administering to the subject a therapeutically effective amount of an FXR agonist.

2. The method of claim 1 wherein the FXR agonist is a compound of Formula (II) 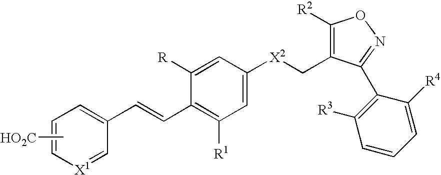
wherein X¹ is CH or N; X² is O or NH; R and R¹ are independently H, lower alkyl, halogen, or CF₃; R² is lower alkyl; R³ and R⁴ are independently H, lower alkyl, halogen, CF₃, OH, O-alkyl, or O-polyhaloalkyl.

3. The method of claim 1 wherein the FXR agonist comprises a compound of Formula (I):

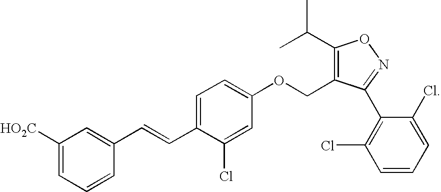

4. A method of reducing or preventing development of liver fibrosis comprising administering to a mammalian subject in need of such treatment a therapeutically effective amount of an FXR agonist.

5. The method of claim 4 wherein the FXR agonist comprises a compound of Formula (II) 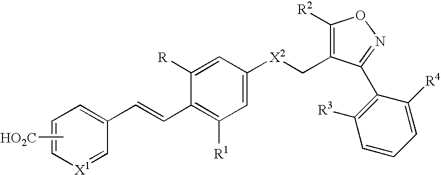
wherein X¹ is CH or N; X² is O or NH; R and R¹ are independently H, lower alkyl, halogen, or CF₃; R² is lower alkyl; R³ and R⁴ are independently H, lower alkyl, halogen, CF₃, OH, O-alkyl, or O-polyhaloalkyl.

6. The method of claim 4 wherein the FXR agonist comprises a compound of Formula (I):

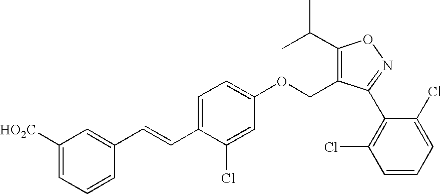

7. A method according to claim 1 where said FXR agonist is not a naturally occurring bile acid.

8. A method according to claim 4 where said FXR agonist is not a naturally occurring bile acid.

9. A method according to claim 1 where said FXR agonist is a synthetic small molecule organic compound.

10. A method according to claim 4 where said FXR agonist is a synthetic small molecule organic compound.

11. A method according to claim 9 where a naturally occuring bile acid is administered concurrently with said FXR agonist.

12. A method according to claim 10 where a naturally occuring bile acid is administered concurrently with said FXR agonist.
